(19) United States
(12) Patent Application Publication
(10) Pub. No.: US 2016/0001044 A1
(43) Pub. Date: Jan. 7, 2016

(54) PIEZOELECTRIC STEERING FOR CATHETERS AND PULL WIRES

(71) Applicant: Siemens Medical Solutions USA, Inc., Malvern, PA (US)

(72) Inventor: John Christopher Rauch, Mesa, AZ (US)

(21) Appl. No.: 14/323,016

(22) Filed: Jul. 3, 2014

(51) Int. Cl. A61M 25/01; H01L 41/09; H01L 41/08

(52) U.S. Cl. CPC A61M 25/0158; H01L 41/082; H01L 41/0926; A61M 25/0147

(57) ABSTRACT

A steerable body insertion device is provided that includes a bendable non-piezoelectric element configured to move within patient anatomy. The non-piezoelectric element extends an element length between a proximal end and a distal end and having an element center axis extending along the element length when the non-piezoelectric element is in a non-bent state. The insertion device also includes a first piezoelectric strand coupled to a surface of the non-piezoelectric element and extending a first strand length. The first piezoelectric strand has a first strand center axis extending substantially parallel to the element center axis along the first stand length. When a first voltage is applied to the first piezoelectric strand, the first piezoelectric strand is configured to contract and cause the non-piezoelectric element to bend away from the element center axis.

### TECHNOLOGY FIELD

The present application relates generally to steerable body insertion devices, such as catheters and pull wires, and in particular, to steerable body insertion devices having piezoelectric elements coupled thereto for steering of the devices.

### BACKGROUND

Conventional body insertion devices, such as catheters, and methods for navigating the body insertion devices inside the vasculature of bodies include mechanical deflection of the catheter and remote magnetic navigation (RMN). Mechanical deflection typically includes having two wires (e.g., pull wires) that extend through the catheter to a steerable or distal end of the catheter where they are bound together at a fixed point. The mechanical deflection catheter bends when one pull wire is pulled in relation to the other. Mechanical deflection catheters typically restrict the motion of the bend by having a rigid tube or other structure that binds the wires together through the non-bending portion of the catheter.

RMN includes a catheter having one or more magnets into the steerable end of the catheter and uses external magnetic fields to cause the deflection in the device. RMN generally operates by using two large magnets placed on either side of the patient and alterations in the magnetic field produced by the magnets deflects the tips of catheters within the patient to the desired direction. Although body insertion devices and methods for navigating the body insertion devices exist, there is a continuing need for more and different body insertion devices and methods for navigating the devices.

### SUMMARY

Embodiments provide a steerable body insertion device that includes a bendable non-piezoelectric element configured to move within patient anatomy. The non-piezoelectric element extends an element length between a proximal end and a distal end and having an element center axis extending along the element length when the non-piezoelectric element is in a non-bent state. The insertion device also includes a first piezoelectric strand coupled to a surface of the non-piezoelectric element and extending a first strand length. The first piezoelectric strand has a first strand center axis extending substantially parallel to the element center axis along the first stand length. When a first voltage is applied to the first piezoelectric strand, the first piezoelectric strand is configured to contract and cause the non-piezoelectric element to bend away from the element center axis.

According to an embodiment, the bendable non-piezoelectric element is a pull wire and the first piezoelectric strand is coupled to a pull wire surface at the distal end.

According to another embodiment, the bendable non-piezoelectric element is a catheter and the first piezoelectric strand is coupled to a catheter surface.

In one embodiment, the steerable body insertion device further includes a second piezoelectric strand coupled to the non-piezoelectric element, opposing the first piezoelectric strand, having a second strand length, and having a second strand center axis extending along the second stand length and substantially parallel to the element center axis. When a first voltage is applied to the first piezoelectric strand, the first piezoelectric strand is configured to contract and cause the non-piezoelectric element to bend away from the element center axis in a first direction toward the first piezoelectric strand relative to the element axis. When a second voltage is applied to the second piezoelectric strand, the second piezoelectric strand is configured to contract and cause the non-piezoelectric element to bend away from the element center axis in a second direction toward the second piezoelectric strand relative to the element center axis, the second direction being opposite the first direction.

In one embodiment, the steerable body insertion device further includes a second piezoelectric strand coupled to the surface of the bendable non-piezoelectric element and spaced from the first piezoelectric strand. The second piezoelectric strand extends a second strand length and has a second strand center axis extending substantially parallel to the element center axis along the second stand length. When a second voltage is applied to the second piezoelectric strand simultaneously with the first voltage applied to the first piezoelectric strand, the first piezoelectric strand and the second piezoelectric strand are each configured to contract and cause the bendable non-piezoelectric element to bend away from the element center axis in a third direction, the third direction having directional components in the first direction and the second direction.

In yet another embodiment, the steerable body insertion device further includes a second piezoelectric strand coupled to the surface of the bendable non-piezoelectric element and spaced from the first piezoelectric strand. The second piezoelectric strand extends a second strand length and has a second strand center axis extending substantially parallel to the element center axis along the second stand length. The steerable body insertion device further includes a third piezoelectric strand coupled to the surface of the bendable non-piezoelectric element and spaced from the first and second piezoelectric strands. The third piezoelectric strand extends a third strand length and has a third strand center axis extending substantially parallel to the element center axis along the third stand length. When a second voltage is applied to the second piezoelectric strand, the second piezoelectric strand is configured to contract and cause the bendable non-piezoelectric element to bend away from the element center axis in a second direction, the second direction being different than the first direction. When a third voltage is applied to the third piezoelectric strand, the third piezoelectric strand is configured to contract and cause the bendable non-piezoelectric element to bend away from the element center axis in a third direction, the third direction being different than the first direction and the second direction.

In one aspect of an embodiment, the amount that the bendable non-piezoelectric element bends away from the element center axis is proportional to the magnitude of the first voltage applied to the first piezoelectric strand.

Embodiments provide a steerable body insertion device that includes a bendable non-piezoelectric element configured to move within patient anatomy. The bendable non-piezoelectric element extends an element length between a proximal end and a distal end. The bendable non-piezoelectric element has an element center axis extending the element length when the non-piezoelectric element is in a non-bent element state. The body insertion device also includes one or more piezoelectric strands embedded within the non-piezoelectric element. The one or more piezoelectric strands extend a strand length between corresponding strand ends and have a corresponding strand center axis extending substantially parallel to the element center axis along the strand length when the corresponding piezoelectric strand is in a non-bent strand state. When a voltage is applied to the one or more piezoelectric strands, the one or more piezoelectric strands are configured to contract and cause the non-piezoelectric element to bend away from the element center axis in a direction toward the one or more piezoelectric strands relative to the element center axis.

According to an embodiment, the steerable body insertion device further includes a plurality of piezoelectric strands each having a corresponding strand center axis that is spaced equidistant from the element center axis. When the voltage is applied to the one or more piezoelectric strands, the one or more piezoelectric strands are configured to contract and cause the non-piezoelectric element to bend away from the element center axis in a direction toward the one or more piezoelectric strands relative to the element center axis.

According to an embodiment, the steerable body insertion device further includes an inner lumen portion extending along the element length. The non-piezoelectric element includes an outer portion at least partially housing the inner lumen portion. Each of the plurality of piezoelectric strands of the combined piezoelectric strand set are spaced from each other and disposed within the outer portion. When the voltage is applied to the one or more piezoelectric strands, the one or more piezoelectric strands are configured to contract and cause the outer portion of the non-piezoelectric element to bend away from the element center axis in a direction toward the one or more piezoelectric strands relative to the element center axis.

In one embodiment, the plurality of piezoelectric strands includes a first piezoelectric strand, a second piezoelectric strand and a third piezoelectric strand each having a corresponding strand center axis spaced equidistant from each other.

In another embodiment, the bendable non-piezoelectric element is a portion of a sheath.

In yet another embodiment, the bendable non-piezoelectric element is a portion of a catheter.

According to one embodiment, the steerable body insertion device further includes a first piezoelectric strand and a second piezoelectric strand. When a first voltage is applied to the first piezoelectric strand simultaneously with a second voltage applied to the second piezoelectric strand, the first piezoelectric strand and the second piezoelectric strand are each configured to contract and cause the bendable non-piezoelectric element to bend away from the element center axis in a direction toward the one or more piezoelectric strands relative to the element axis. The direction has directional components in a first direction toward the first piezoelectric strand relative to the element axis and a second direction toward the second piezoelectric strand relative to the element center axis.

According to another embodiment, the amount that the bendable non-piezoelectric element bends away from the element center axis is based on the magnitude of the voltage applied to the one or more piezoelectric strands.

In an aspect of an embodiment, the steerable body insertion device further includes multiple sets embedded and substantially centered within the non-piezoelectric element extending along the element length.

In one embodiment, the one or more piezoelectric strands include a plurality of electrodes to separate the one or more piezoelectric strands into a plurality of sub-piezoelectric strands between the electrodes. Each of the one or more sub-piezoelectric strands are configured to contract and cause the non-piezoelectric element to bend away from the element center axis in a direction toward the one or more sub-piezoelectric strands relative to the element center axis when the voltage is applied to a corresponding sub-piezoelectric strand.

In another aspect of an embodiment, a first strand length of a first piezoelectric strand is different than a second strand length of a second piezoelectric strand.

Embodiments provide a system for controlling a steerable body insertion device. The system includes a steerable body insertion device. The steerable body insertion device includes a bendable non-piezoelectric element configured to move within patient anatomy. The bendable non-piezoelectric element extends an element length between a proximal end and a distal end. The bendable non-piezoelectric element has an element center axis extending the element length when bendable the non-piezoelectric element is in a non-bent element state. The steerable body insertion device also includes a plurality of piezoelectric strands coupled to the non-piezoelectric element. Each of the plurality of piezoelectric strands extends a strand length between corresponding strand ends and has a corresponding strand center axis extending substantially parallel to the element center axis along the strand length when the corresponding piezoelectric strand is in a non-bent strand state. Each of the plurality of piezoelectric strands is configured to contract when receiving a voltage and cause the non-piezoelectric element to bend away from the element center axis in a direction toward one or more of the plurality of piezoelectric strands relative to the element center axis. The system also includes a voltage applicator configured to apply the voltage to the one or more piezoelectric strands and a controller configured to control the bend of the bendable non-piezoelectric element by causing the voltage applicator to apply the voltage to the one or more piezoelectric strands.

According to an embodiment, the controller is further configured to control an amount of the bend of the non-piezoelectric element by controlling the voltage applicator to apply a voltage magnitude to the one or more piezoelectric strands.

According to another embodiment, the controller is further configured to control the direction of the bend of the non-piezoelectric element by controlling the voltage applicator to apply a voltage to the one or more piezoelectric strands.

In one embodiment, the voltage applicator is further configured to apply a first voltage to a first piezoelectric strand and simultaneously apply a second voltage to a second piezoelectric strand. The controller is further configured to cause the non-piezoelectric element to bend in a direction having directional components in a first direction toward the first piezoelectric strand relative to the element axis and a second direction toward the second piezoelectric strand relative to the element center axis.

In an aspect of an embodiment, the controller is further configured to cause the magnitude of the second voltage applied to the second piezoelectric strand to be different than the first voltage applied to the first piezoelectric strand.

Embodiments provide a piezoelectric strand set for use with a non-piezoelectric element configured to move within patient anatomy. The piezoelectric strand combination includes a plurality of piezoelectric strands extending a corresponding strand length between corresponding strand ends and having a corresponding strand center axis extending along the corresponding strand length when the plurality of piezoelectric strands piezoelectric strands are in a non-bent state. Each of the plurality of piezoelectric strands strand has opposing electrical contacts electrically connected at opposite ends of each strand configured to receive an applied voltage. Each of the plurality of piezoelectric strands is configured to be embedded into the non-piezoelectric element. When the voltage is applied to one or more of the plurality of piezoelectric strands, the one or more piezoelectric strands are configured to contract and cause the non-piezoelectric element to bend away from a center axis of the non-piezoelectric element in a direction toward the one or more piezoelectric strands relative to the center axis of the non-piezoelectric element.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1A is a side view of a piezoelectric strand coupled to a non-piezoelectric element in a non-bent state when no voltage is applied to the piezoelectric strand for use with exemplary embodiments disclosed herein;

FIG. 1B is a side view of the piezoelectric strand and the non-piezoelectric element 104 in a bent state for use with exemplary embodiments disclosed herein;

FIG. 2 is a chart illustrating different exemplary configurations of one or more piezoelectric strands coupled to a non-piezoelectric element for use with embodiments disclosed herein;

FIG. 3A is a side view of an exemplary piezoelectric strand coupled to a surface of a non-piezoelectric element portion at a distal end of a pull wire according to an exemplary embodiment;

FIG. 3B is a close-up side view of the distal end of the pull wire illustrating the piezoelectric strand coupled to the non-piezoelectric element portion shown in FIG. 3A;

FIG. 4 is a chart showing different exemplary configurations of multiple piezoelectric strands for use with exemplary embodiments disclosed herein;

FIG. 5A is a side view of a single piezoelectric strand embedded in an outer non-piezoelectric element portion at a distal end of a sheath according to an exemplary embodiment;

FIG. 5B is a close-up side view of the piezoelectric strand embedded in the outer non-piezoelectric element portion shown in FIG. 5A and includes cross sectional views along the sheath 502 illustrating a single piezoelectric strand embedded in the sheath;

FIG. 6A is a side view of sheath illustrating one piezoelectric strand of multiple piezoelectric strands embedded in an outer non-piezoelectric element portion at a distal end of the sheath according to an exemplary embodiment;

FIG. 6B is a close-up side view of the sheath shown in FIG. 6A illustrating the one piezoelectric strand embedded in the outer non-piezoelectric element portion and includes cross sectional views along the sheath illustrating three piezoelectric strands embedded in the sheath;

FIG. 7A is a side view of piezoelectric strands embedded and substantially centered in a non-piezoelectric element portion of a catheter according to an exemplary embodiment;

FIG. 7B is a close-up side view of the piezoelectric strand embedded in the non-piezoelectric element portion shown in FIG. 7A and includes cross sectional views along the catheter illustrating three piezoelectric strands embedded and substantially centered in the non-piezoelectric element portion of the catheter;

FIG. 8 shows side views, close-up views and cross sectional views illustrating multiple sets of piezoelectric strands embedded and substantially centered in a non-piezoelectric element portion of a catheter according to an exemplary embodiment;

FIG. 9 shows side views, close-up views and cross sectional views illustrating a piezoelectric strand separated into sub-piezoelectric strands between electrodes according to an exemplary embodiment;

FIG. 10 is a diagram illustrating a system for controlling a steerable body insertion device according to an exemplary embodiment; and

FIG. 11 illustrates an example of a computing environment within which embodiments of the invention may be implemented.

### DETAILED DESCRIPTION OF EXEMPLARY EMBODIMENTS

As described above, mechanical deflection catheters bend when one wire is pulled in relation to the other. For example, pulling one wire relative to another shortens the pulled wire, thereby deflecting or bending the catheter toward the side of the shortened or pulled wire. Some conventional mechanical deflection catheters may bend 180 degrees or even more. Typically, navigating a body includes many twists and turns. Each time mechanical deflection catheters bend to navigate the twists and turns, stress is applied to the wire, limiting the amount of control a user may have on the distal end of the catheter. Also, the rigid structures of conventional mechanical deflection catheters limit the flexibility of the catheter. While some mechanical deflection catheters use robotics to control the deflection by using robotics to move the mechanism which pulls the wires, use of robotics to control these mechanical deflection catheters does not overcome the shortcomings of these conventional mechanical deflection catheters.

As described above, RMN includes large magnets placed on either side of the patient. Alterations in the magnetic fields produced by the magnets deflect the tips of catheters within the patient to the desired direction. Accordingly, systems using RMN may be large and costly. Further, these external systems and components may impede on the angulation of the x-ray system that is used during the study to do the imaging of the device in the patient's vascular anatomy.

Embodiments of the present invention provide piezoelectric strands coupled to non-piezoelectric elements, causing the non-piezoelectric element to bend when voltages are applied to the piezoelectric strands. Embodiments of the present invention provide steerable body insertion devices having piezoelectric strands that cause non-piezoelectric element portions of the steerable body insertion devices to bend wherein when voltages are applied to the piezoelectric strands. In some embodiments, piezoelectric strands may be coupled to a surface of the non-piezoelectric element portions. In other embodiments, piezoelectric strands may be embedded within the non-piezoelectric element portions of steerable body insertion devices.

Piezoelectric strands are not, however, limited to use with steerable body insertion devices. Embodiments may include piezoelectric strands configured to cause flex or bending of an arm or other appendages. This approach may be used in robotic applications to create mechanical appendages to enable interaction with the environment (e.g., for placing or soldering components onto circuit boards) or to enable locomotion of a robot (e.g., to produce snake-like or fish-like movement).

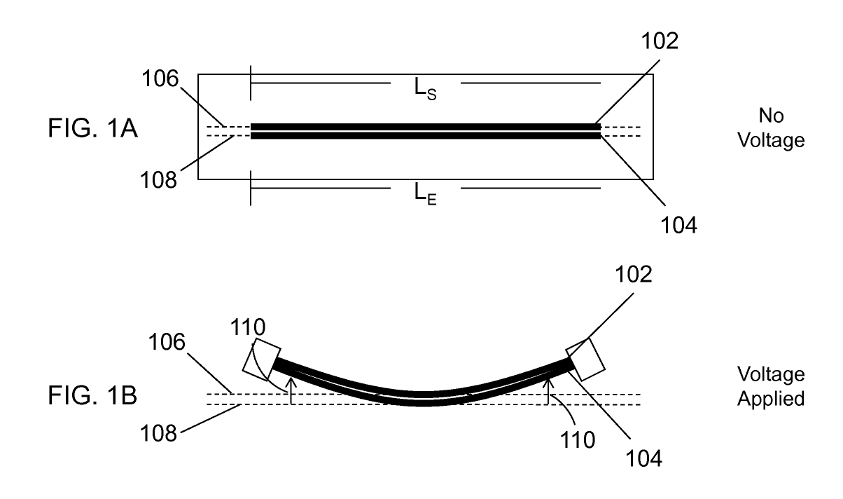

FIG. 1A is a side view of a piezoelectric strand 102 coupled to a non-piezoelectric element 104 in a non-bent state when no voltage is applied to the piezoelectric strand 102 for use with embodiments disclosed herein. The piezoelectric strand 102 may include any type of piezoelectric material having piezoelectric properties and configured to contract when a voltage is applied to the piezoelectric material. Non-piezoelectric element 104 may include any type of non-piezoelectric material. In some embodiments, non-piezoelectric elements may include insulating materials.

FIG. 1B is a side view of the piezoelectric strand 102 and the non-piezoelectric element 104 in a bent state for use with embodiments disclosed herein. When a voltage is applied to the piezoelectric strand 102, the piezoelectric strand 102 is configured to contract and cause the non-piezoelectric element 104, which is coupled to the piezoelectric strand 102 to bend. The amount of bend shown in FIG. 1B is merely exemplary. Embodiments may include piezoelectric strands configured to bend in amounts different than the amount of bend shown in FIG. 1B depending on the properties of the piezoelectric material and the amount of applied voltage.

Embodiments may include piezoelectric materials configured to have axes where the effects of contraction are observed. For example, as shown in FIG. 1A and FIG. 1B, piezoelectric strand 102 may include a strand center axis 106 extending along the strand length L_{S}. Non-piezoelectric element 104 may include an element center axis 108 extending along the element length L_{E}. As shown, the first strand center axis 106 extends substantially parallel to the element center axis 108. In the embodiment shown in FIG. 1B, piezoelectric strand 102 is configured to contract and cause the non-piezoelectric element 104 to bend away from the element center axis 108 in a first direction, indicated by arrows 110, toward the piezoelectric strand 102 relative to the element center axis 108. The amount that the bendable non-piezoelectric element 104 bends away from the element center axis 108 may be proportional to the magnitude of the voltage applied to the piezoelectric strand 102. Embodiments may include piezoelectric strands configured such that effects of the contractions are not observed along center axes of the strands.

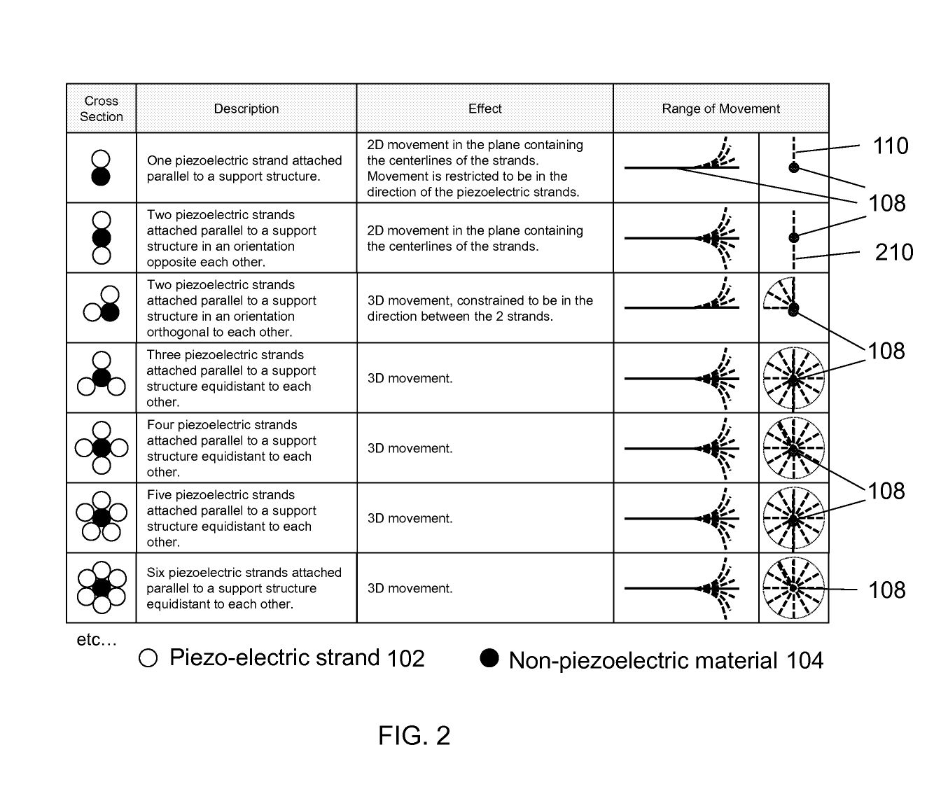

FIG. 2 is a chart illustrating different exemplary configurations of one or more piezoelectric strands 102 coupled to a non-piezoelectric element 104 for use with embodiments disclosed herein. Each row in FIG. 2 corresponds to a different configuration of one or more piezoelectric strands 102 coupled to a non-piezoelectric element 104. For example, the first row corresponds to a configuration of one piezoelectric strand 102 coupled to a non-piezoelectric element 104 and the second row corresponds to a configuration of two piezoelectric strands 102 coupled to a non-piezoelectric element 104. The first column of FIG. 2 illustrates cross sectional views of the different configurations of the piezoelectric strands 102. The second column of FIG. 2 includes brief descriptions of the different configurations of the piezoelectric strands 102. The third column of FIG. 2 describes the dimensional movement of the different configurations of the piezoelectric strands 102. The fourth and fifth columns of FIG. 2 illustrate exemplary ranges of movement of the different configurations of the piezoelectric strands 102. The fourth column of FIG. 2 includes side views showing the range of movement and the fifth column includes cross sectional views showing the range of movement of the non-piezoelectric element 104.

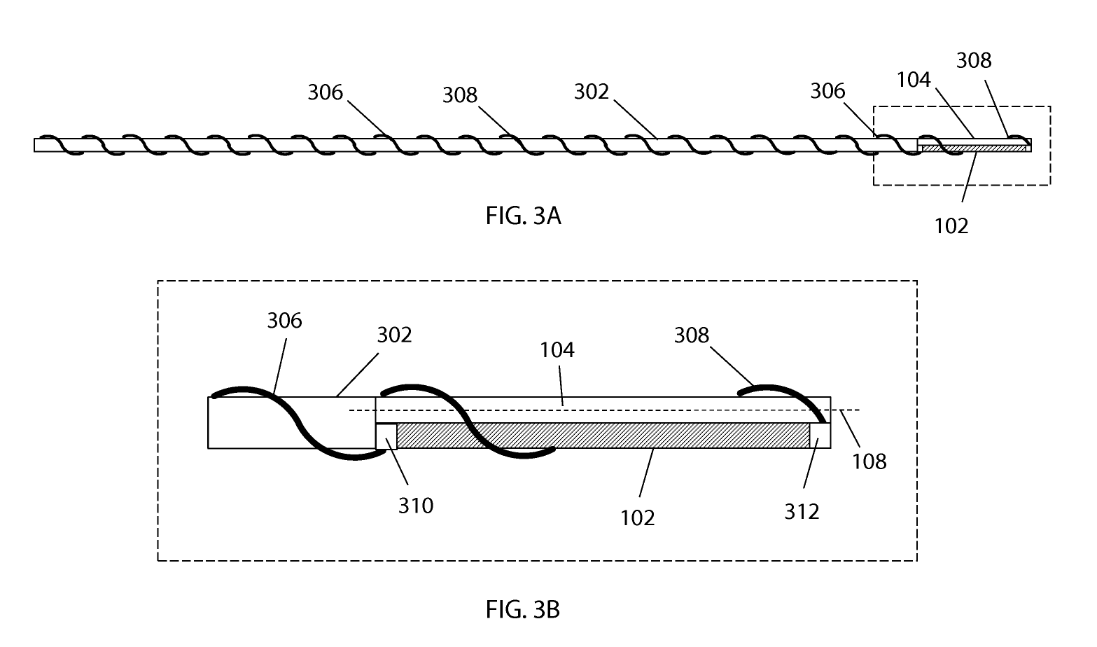

Each configuration of the piezoelectric strands 102 shown in FIG. 2 may be used to bend a non-piezoelectric element portion 104 of a steerable body insertion device, such as pull wire 302, shown in FIG. 3A and FIG. 3B. Each non-piezoelectric element 104 shown in FIG. 2 may extend an element length L_{E} (shown in FIG. 1A) between a proximal end and a distal end. Each non-piezoelectric element 104 may also have an element center axis 108 extending along the element length L_{E} when the non-piezoelectric element 104 is in a non-bent state. Each of the piezoelectric strands 102 shown in FIG. 2 may extend a corresponding strand length L_{S} (shown in FIG. 1A) between corresponding proximal ends and distal ends. Each of the piezoelectric strands 102 shown in FIG. 2 may have a corresponding strand center axis 106 (shown in FIG. 1A) extending along the corresponding strand length L_{S} when the plurality of piezoelectric strands 102 are in a non-bent state

As shown in FIG. 2, each piezoelectric strand 102 may be coupled to a surface of a corresponding non-piezoelectric element 104. For example, as shown in the first row of FIG. 2, one piezoelectric strand 102 may be coupled to a surface of the non-piezoelectric element 104. When a first voltage is applied to the piezoelectric strand 102, the piezoelectric strand 102 is configured to contract and cause the non-piezoelectric element 104 to bend away from the element center axis 108. As shown in the range of movement columns, the non-piezoelectric element 104 may have 2-dimensional (2D) movement. The movement is shown in the plane that includes the strand center axis 106 (shown in FIG. 1A) and in the direction of the piezoelectric strands 102.

Referring to the second row of FIG. 2, two piezoelectric strands 102 may be coupled to the surface of the non-piezoelectric element 104 and oppose each other such that the element center axis 108 and the corresponding strand center axes 106 (shown in FIG. 1A) are in the same plane. When a first voltage is applied to a first piezoelectric strand 102 (e.g., top strand), the first piezoelectric strand 102 may be configured to contract and cause the non-piezoelectric element 104 to bend away from the element center axis 108 in a first direction 110 (upward direction shown in the fifth column of the second row) toward the first piezoelectric strand 102 relative to the element axis 108. When a second voltage is applied to a second piezoelectric strand 102 (e.g., bottom strand), the second piezoelectric strand 102 may be configured to contract and cause the non-piezoelectric element to bend away from the element center axis 108 in a second direction 210 (downward direction shown in the fifth column of the second row) opposite the first direction and toward the second piezoelectric strand 102 relative to the element center axis 108. In this configuration, the non-piezoelectric element 104 also has 2D movement in the plane that includes the center axes 106 (shown in FIG. 1A) of the first and second piezoelectric strands 102.

Referring to the third row of FIG. 2, two piezoelectric strands 102 may be coupled to the surface of the non-piezoelectric element 104. As shown, a first piezoelectric strand 102 (e.g., top strand) and a second piezoelectric strand 102 (e.g., left side strand) may be coupled to the surface of the non-piezoelectric element 104. When a first voltage is applied to the first piezoelectric strand 102 (e.g., top strand), the first piezoelectric strand 102 may be configured to contract and cause the non-piezoelectric element 104 to bend away from the element center axis 108 in a first direction (upward direction shown in the fifth column of the third row) toward the first piezoelectric strand 102 relative to the element axis 108. When a second voltage is applied to a second piezoelectric strand 102 (e.g., left side strand), the second piezoelectric strand 102 may be configured to contract and cause the non-piezoelectric element to bend away from the element center axis 108 in a second direction (direction to the left shown in the fifth column of the third row) toward the second piezoelectric strand 102 relative to the element center axis 108. These first and second directions, however, are not in the same plane that includes the center axes 106 of the first and second piezoelectric strands 102. Therefore, in this configuration, the combination of varying voltages applied to both piezoelectric strands enables the non-piezoelectric element 104 to experience 3-dimensional (3D) movement.

In addition to the movement of the non-piezoelectric element 104 in the first direction toward the first strand 102 and the movement in the second direction toward the second strand 102, the non-piezoelectric element 104 may also move in directions between the first and second piezoelectric strands 102. For example, when the second voltage is applied to the second piezoelectric strand 102 simultaneously with the first voltage applied to the first piezoelectric strand 102, the first piezoelectric strand 102 and the second piezoelectric strand 102 are each configured to contract and cause the non-piezoelectric element 104 to bend away from the element center axis 108 in a third direction between the first and second strands 102. That is, the third direction includes directional components in the first direction and the second direction. Further, the precise direction of the movement between the first and second strands 102 may be based on a magnitude of the voltage applied to the first piezoelectric strand 102. For example, if the magnitude of the voltage applied to the first piezoelectric strand 102 is greater than the magnitude of the voltage applied to the second piezoelectric strand 102, the direction of the movement of the non-piezoelectric element 104 may be closer to the first direction toward the first strand 102 than the second direction toward the second strand 102.

Referring to the fourth row of FIG. 2, three piezoelectric strands 102 may be coupled to the surface of the non-piezoelectric element 104. As shown, a first piezoelectric strand 102 (e.g., top strand), a second piezoelectric strand 102 (e.g., bottom left strand) may be coupled to the surface of the non-piezoelectric element 104 and a third piezoelectric strand 102 (e.g., bottom right strand) may be coupled to the surface of the non-piezoelectric element 104. When a voltage is applied to any one of the three piezoelectric strands 102, any one of the piezoelectric strands 102 may be configured to contract and cause the non-piezoelectric element 104 to bend away from the element center axis 108 in the direction of the corresponding piezoelectric strand 102.

In addition, when voltages are applied simultaneously to two of the three piezoelectric strands 102, the two piezoelectric strands 102 may be configured to contract and cause the non-piezoelectric element 104 to bend away from the element center axis 108 in directions between the first and second piezoelectric strands 102. Accordingly, in this configuration, the non-piezoelectric element 104 has 3-dimensional (3D) movement in any direction as shown in column five.

Embodiments may include any additional number of piezoelectric strands 102 (e.g., 4 strands, 5 strands, 6 strands, etc.) as shown as shown in rows 5, 6 and 7, respectively, in FIG. 2. In these configurations, the non-piezoelectric element 104 may also have 3-dimensional (3D) movement in any direction. The determination of how many piezoelectric strands 102 are used may include various factors. For example, while each additional piezoelectric strand 102 may offer more control of movement of the non-piezoelectric element 104, each additional piezoelectric strand 102 includes an additional two wires to apply the corresponding voltages, which takes up space additional space.

FIG. 3A is a side view of an exemplary piezoelectric strand 102 coupled to a surface of a non-piezoelectric element portion 104 at a distal end of a pull wire 302 according to an exemplary embodiment. FIG. 3B is a close-up side view of the distal end of the pull wire 302 illustrating the piezoelectric strand 102 coupled to the non-piezoelectric element portion 104 shown in FIG. 3A. Embodiments may include piezoelectric strands coupled to surfaces of any type of body insertion devices, such as catheters and sheaths.

As shown in FIG. 3A and FIG. 3B, a first conductor 306 may wrap around pull wire 302 and be electrically connected to piezoelectric strand 102 via a first electrical contact 310. Further, a second conductor 308 may wrap around pull wire 302 and be electrically connected to piezoelectric strand 102 via second electrical contact 312. The size, shape and location of the conductors 306 and 308 in the embodiment shown in FIG. 3A and FIG. 3B are merely exemplary. Embodiments may include different configurations of coupling conductors to steerable body insertion devices and electrically connecting to piezoelectric strands. Embodiments may include any type of conductor configured to allow electricity to flow.

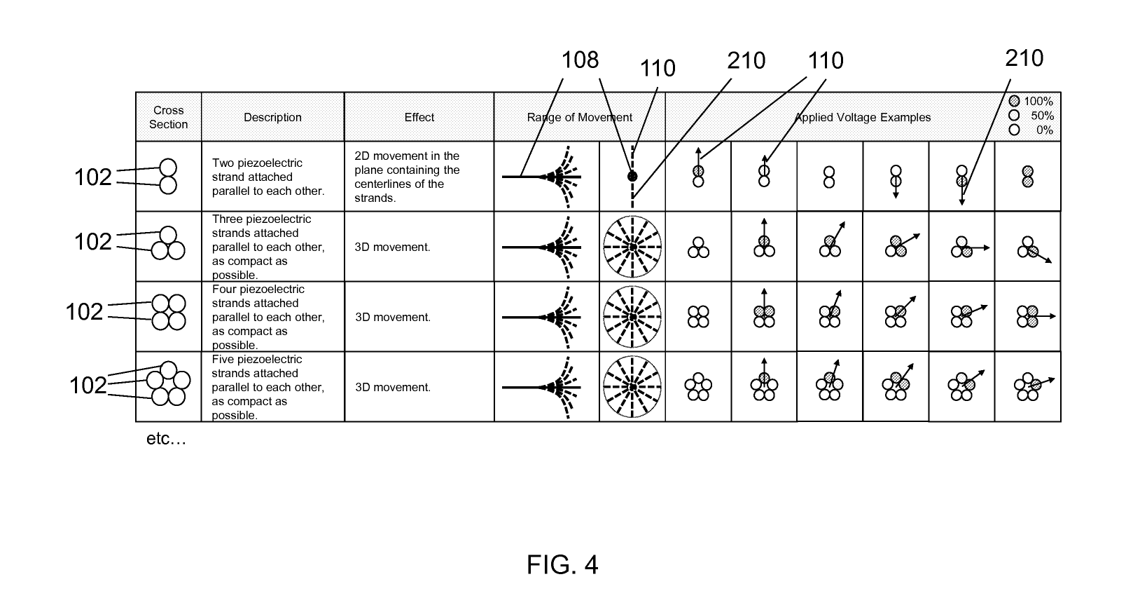

FIG. 4 is a chart showing different exemplary configurations of multiple piezoelectric strands 102 for use with embodiments disclosed herein. Each row in FIG. 4 corresponds to a different configuration of multiple piezoelectric strands 102. For example, the first row corresponds to a configuration of two multiple piezoelectric strands 102 and the second row corresponds to a configuration of three multiple piezoelectric strands 102. The first column of FIG. 4 illustrates cross sectional views of the different configuration of multiple piezoelectric strands 102. The second column of FIG. 4 includes brief descriptions of the different configurations of multiple piezoelectric strands 102. The third column of FIG. 4 describes the dimensional movement of the different configurations of multiple piezoelectric strands 102. The fourth and fifth columns of FIG. 4 illustrate exemplary ranges of movement of the different configurations of multiple piezoelectric strands 102. The sixth through eleventh columns of FIG. 4 illustrate exemplary directions of movement of the multiple piezoelectric strands 102 responsive to different amounts of voltages applied to corresponding multiple piezoelectric strands 102.

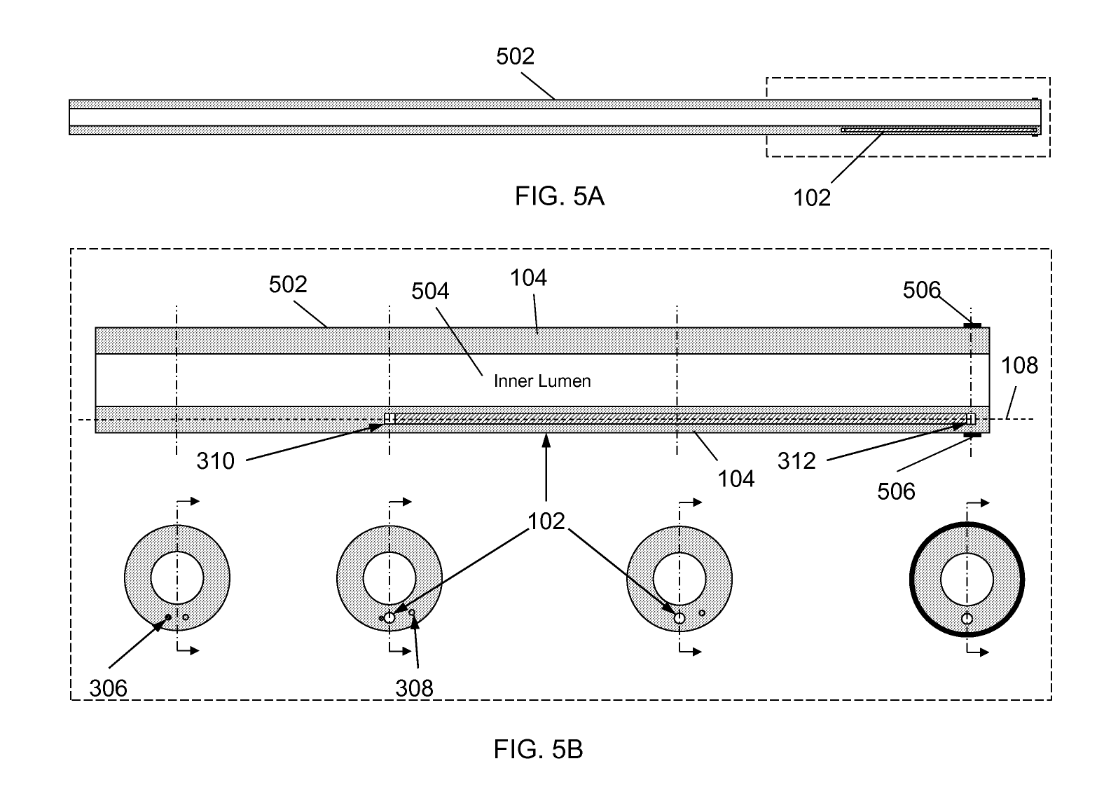

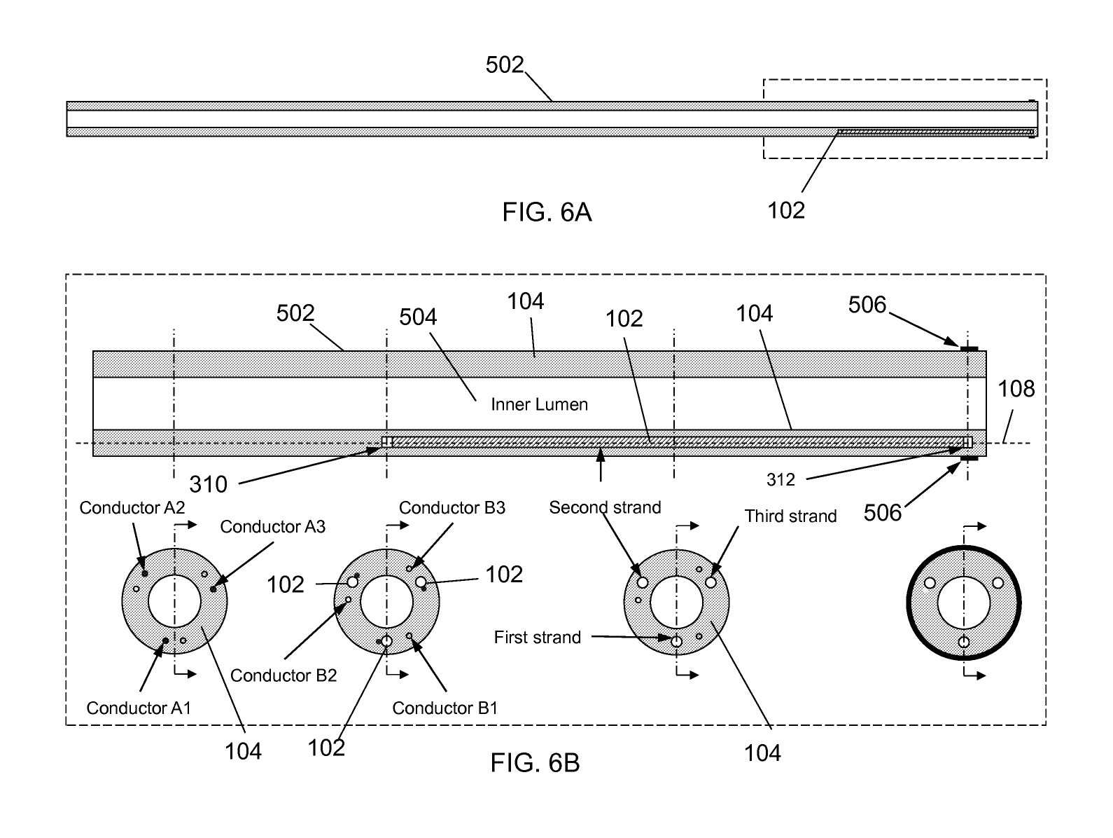

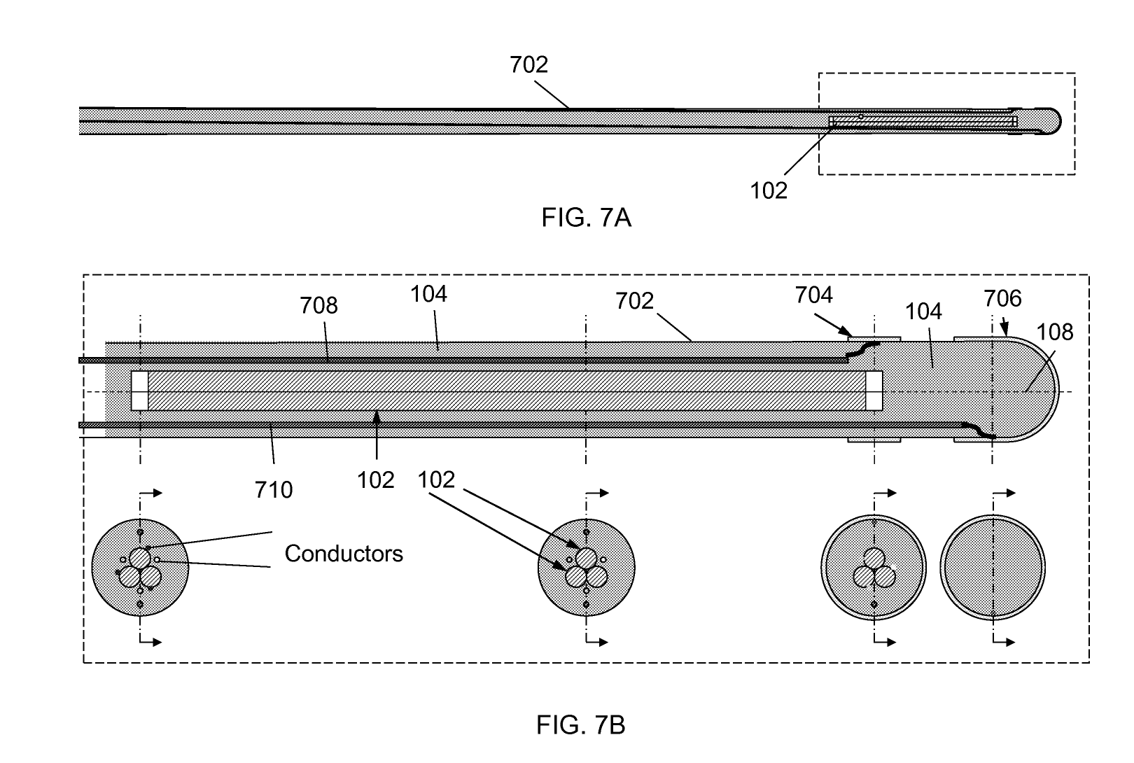

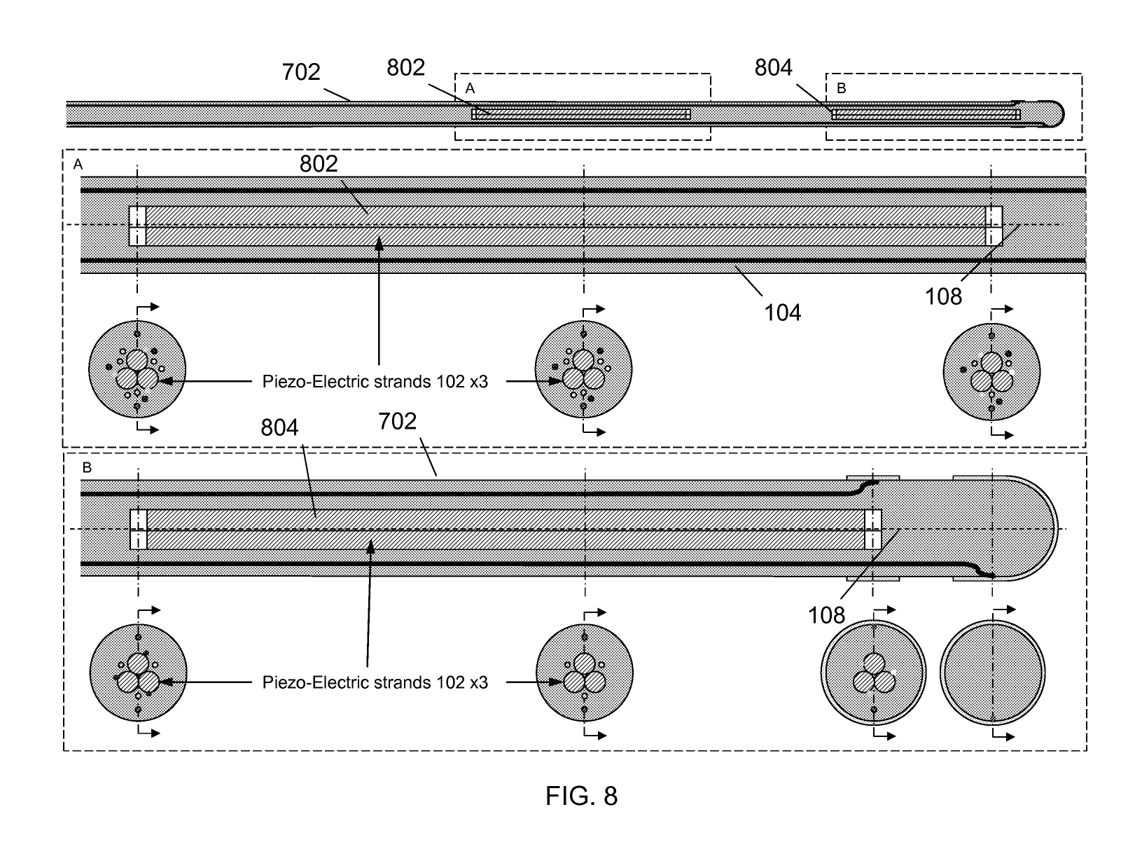

Each configuration of multiple piezoelectric strands 102 shown in FIG. 4 may be used with a non-piezoelectric element configured to move within patient anatomy. For example, each piezoelectric strand configuration may be embedded within and used to bend a non-piezoelectric element portion of a steerable body insertion device, such as a micro-catheter or sheath shown in FIG. 5A, FIG. 5B and FIG. 6 or an electrophysiology catheter shown in FIG. 7 and FIG. 8.

Each of the piezoelectric strands 102 shown in FIG. 4 may extend a corresponding strand length L_{S} (shown in FIG. 1A) between corresponding strand ends and have a corresponding strand center axis 106 (shown in FIG. 1A) extending along the corresponding strand length L_{S} when the plurality of piezoelectric strands 102 are in a non-bent state.

When a voltage is applied to the piezoelectric strands 106, the piezoelectric strands 102 are configured to contract. If the piezoelectric strands 102 are embedded within a non-piezoelectric element portion 104, the non-piezoelectric element portion 104 is caused to bend away from a center axis 108 (shown in FIG. 6 through FIG. 8) of the non-piezoelectric element portion 104 in a direction toward the one or more piezoelectric strands 102 relative to the center axis 108 of the non-piezoelectric element.

Referring to the first row of FIG. 4, two piezoelectric strands 102 may oppose each other such that the corresponding strand center axes 106 (shown in FIG. 1A) are in the same plane. When a first voltage is applied to a first piezoelectric strand 102 (e.g., top strand), the first piezoelectric strand 102 may be configured to contract and cause the non-piezoelectric element 104 to bend away from the element center axis 108 in a first direction (upward direction shown in the fifth column of the first row) toward the first piezoelectric strand 102 relative to the element axis 108. In this configuration, the non-piezoelectric element 104 has 2D movement in the plane that includes the center axes 106 (shown in FIG. 1A) of the first and second piezoelectric strands 102.

Referring to the applied voltage examples shown in FIG. 4, the examples show the corresponding different configurations of the piezoelectric strands 102, the direction that the non-piezoelectric element 104 bends from the element center axis 108 and the amount of bend that the non-piezoelectric element 104 bends when voltages are applied. The non-piezoelectric element 104 in which the piezoelectric strands 102 are embedded is not shown in the applied voltage examples at FIG. 4 for simplification. The configurations of the piezoelectric strands 102 shown in FIG. 4 are merely exemplary. For example, the piezoelectric strands 102 shown in FIG. 4 and in FIG. 7 may be coupled closely together. In other embodiments, piezoelectric strands may be spaced from each other, such as for example the piezoelectric strands shown in FIG. 6.

In the applied voltage example in row 1, the amount that the non-piezoelectric element bends away from the element center axis is based on the magnitude of the voltage applied to the piezoelectric strands 102. For example as shown in the first applied voltage example in row 1, a 100% voltage is applied to the top strand 102. In the second applied voltage example in row 1, a 50% voltage is applied to the top strand 102. Accordingly the arrow 110 in the first example is larger than the arrow 110 in the second example, indicating a greater amount of bend by the non-piezoelectric element 104 in the first example.

As shown in the following rows in FIG. 4, three or more piezoelectric strands 102 may be used to cause bend of the non-piezoelectric element 104. In these configurations of three or more piezoelectric strands 102, the non-piezoelectric element 104 may have 3D movement. For example, as shown in row 2 of FIG. 4, a first piezoelectric strand 102, a second piezoelectric strand 102 and a third piezoelectric strand 102 may be configured such that each corresponding strand center axis (strand center axis 102 is shown in FIG. 1) is spaced equidistant from each other. In these configurations of three or more piezoelectric strands 102, when voltages are applied to two different piezoelectric strands 102 simultaneously, the bendable non-piezoelectric element 104 may be caused to bend away from the element center axis 108 in a direction between the two different piezoelectric strands 102. Accordingly, in this configuration, the non-piezoelectric element 104 has 3-dimensional (3D) movement in any direction as shown in the range of movement columns in rows 2-4 in FIG. 4. For example, if equal amounts of voltages are applied to two adjacent piezoelectric strands 102 (e.g., 4^{th} applied voltage example in row 3), the non-piezoelectric element 104 may be caused to bend away from the element center axis 108 in a direction halfway between the two different piezoelectric strands 102. If different amounts of voltages are applied to piezoelectric element 104 may be caused to bend away from the element center axis 108 in a direction closer to the piezoelectric strand 102 having the higher voltage applied thereto.

FIG. 5A is a side view of a piezoelectric strand 102 embedded in an outer non-piezoelectric element portion 104 at a distal end of a sheath 502 according to an exemplary embodiment. FIG. 5B is a close-up side view of the piezoelectric strand 102 embedded in the outer non-piezoelectric element portion 104 shown in FIG. 5A and includes cross sectional views along the sheath 502 illustrating a single piezoelectric strand embedded in the sheath 502. As shown in FIG. 5B, the sheath 502 may include an inner lumen portion 504, an outer non-piezoelectric element portion 104 housing the inner lumen portion 504 and radio opaque markers 506. The cross sectional views illustrate the position of the first conductor 306 and the second conductor 308 embedded in the outer non-piezoelectric element portion 104. In some embodiments, the outer non-piezoelectric element portion 104 may partially house the inner lumen portion 504. FIG. 5A and FIG. 5B illustrate a single strand 102 embedded in the outer non-piezoelectric element portion 104. As shown in FIG. 5B, the piezoelectric strand 102 may include a first electrical contact 310 and second electrical contact 312. When a voltage is applied to the piezoelectric strand 102, the piezoelectric strand 102 may contract and cause the outer non-piezoelectric element portion 104 to bend away from the element center axis 108 in a direction toward the piezoelectric strands 102 relative to the element center axis 108.

FIG. 6A and FIG. 6B also show the sheath in FIGS. 5A and 5B. In FIG. 6A and FIG. 6B, however, three piezoelectric strands 102 are embedded in the outer non-piezoelectric element portion 104 of the sheath 502. As shown in the cross sectional views in FIG. 6B, each strand 102 may be electrically connected to first and second conductors (conductors A1 and B1 for the first strand, conductors A2 and B2 for the first strand and conductors A3 and B3 for the third strand) via corresponding electrical contacts (e.g., electrical contact A1 and B1 for the first strand).

FIG. 7A is a side view of piezoelectric strands 102 embedded and substantially centered in a non-piezoelectric element portion 104 of a catheter 702 according to an exemplary embodiment. FIG. 7B is a close-up side view of the piezoelectric strand 102 embedded in the non-piezoelectric element portion 104 shown in FIG. 7A and includes cross sectional views along the catheter 702 illustrating three piezoelectric strands 102 embedded and substantially centered in the non-piezoelectric element portion 104 of the catheter 702. As shown in the cross sectional views in FIG. 7B, each strand 102 may be electrically connected to first and second conductors (conductors A1 and B1 for the first strand, conductors A2 and B2 for the first strand and conductors A3 and B3 for the third strand) via corresponding electrical contacts (e.g., electrical contact A1 and B1 for the first strand). As shown in FIG. 7B, the catheter 702 may include a ring electrode 704 electrically connected to conductor 708 and a tip electrode 706 electrically connected to conductor 710. As shown in the cross sectional views in FIG. 7B, each strand 102 may be electrically connected to first and second conductors in a similar manner as the conductors A1, B1, A2, B2, A3, B3 shown in FIG. 6B.

FIG. 8 shows side views, close-up views and cross sectional views illustrating multiple sets of piezoelectric strands 102 embedded and substantially centered in a non-piezoelectric element portion 104 of a catheter 702 according to an exemplary embodiment. As shown in FIG. 8, the multiple sets of piezoelectric strands 102 include a first set 802 and a second set 804 spaced from each other along a length of the catheter 702. Embodiments may, however, include sets of piezoelectric strands that are attached to each other.

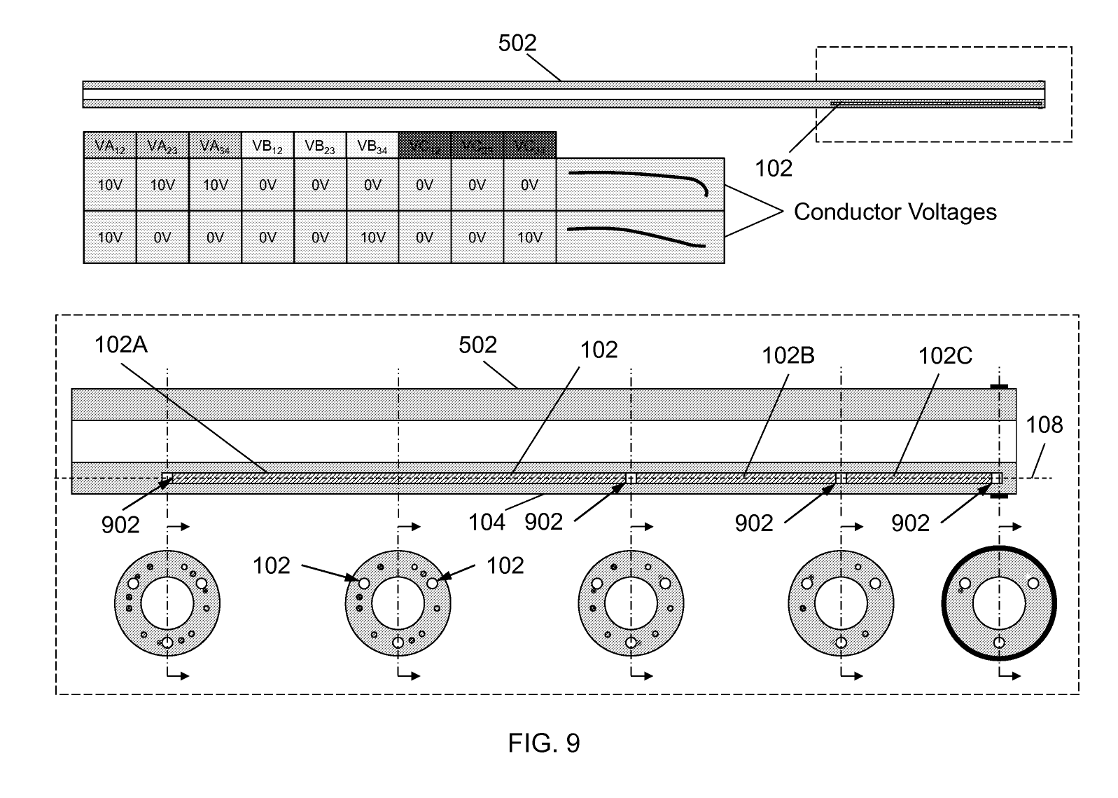

FIG. 9 shows side views, close-up views and cross sectional views illustrating a piezoelectric strand 102 separated into sub-piezoelectric strands 102A, 102B and 102C between electrodes 902 according to an exemplary embodiment. As shown in FIG. 9, each of the sub-piezoelectric strands 102A, 102B and 102C may have different lengths along the length L_{S} (shown in FIG. 1A) of the piezoelectric strand 102. Embodiments may include one or more sub-piezoelectric strands having the same lengths along the length L_{S}. Each sub-piezoelectric strand 102A, 102B and 102C may be independently controllable to contract and cause the non-piezoelectric element 104 to bend away from the element center axis 108 in a direction toward the corresponding sub-piezoelectric strand 102A, 102B and 102C relative to the element center axis 108 when the voltage is applied to a corresponding sub-piezoelectric strand 102A, 102B and 102C.

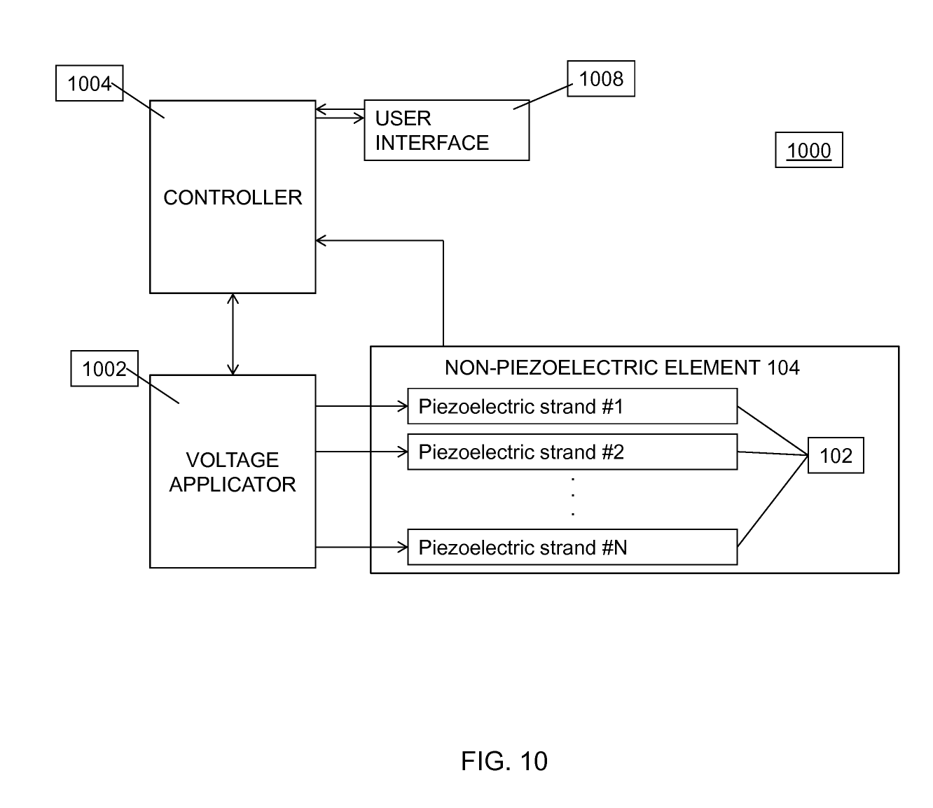

FIG. 10 is a diagram illustrating a system for controlling a steerable body insertion device according to an exemplary embodiment. As shown in FIG. 10, the system 1000 may include a bendable non-piezoelectric element 104, one or more piezoelectric strands 102 coupled to the non-piezoelectric element 104, a voltage applicator 1002 configured to apply voltages to the one or more piezoelectric strands 102, a controller 1004 configured to control the bend of the bendable non-piezoelectric 104 element by causing the voltage applicator 1002 to apply varying voltages to the one or more piezoelectric strands 102. The controller 1004 may control the amount and/or direction of the bend of the non-piezoelectric element by controlling the voltage applicator 1002 to apply a voltage of varying magnitude to the one or more piezoelectric strands 102. The controller 1004 may apply simultaneous voltages to two different piezoelectric strands 102. The controller 1004 may cause the non-piezoelectric element 104 to bend in a direction having directional components in a first direction toward one piezoelectric strand 102 and a second direction toward a second piezoelectric strand 102.

The system 1000 may also include a user interface 1008 configured to communicate with the controller 1004. The user interface may receive instructions from a user (not shown) to control the bend of the non-piezoelectric element 104. The instructions may include amounts of bend (e.g., distances, degrees or radians) and magnitudes of voltages to be applied to the one or more piezoelectric strands 102. The controller 1004 may also receive feedback from the one or more piezoelectric strands 102 and/or the non-piezoelectric element 104 regarding the amount of bend and magnitudes of voltages and may automatically adjust one or more voltages.

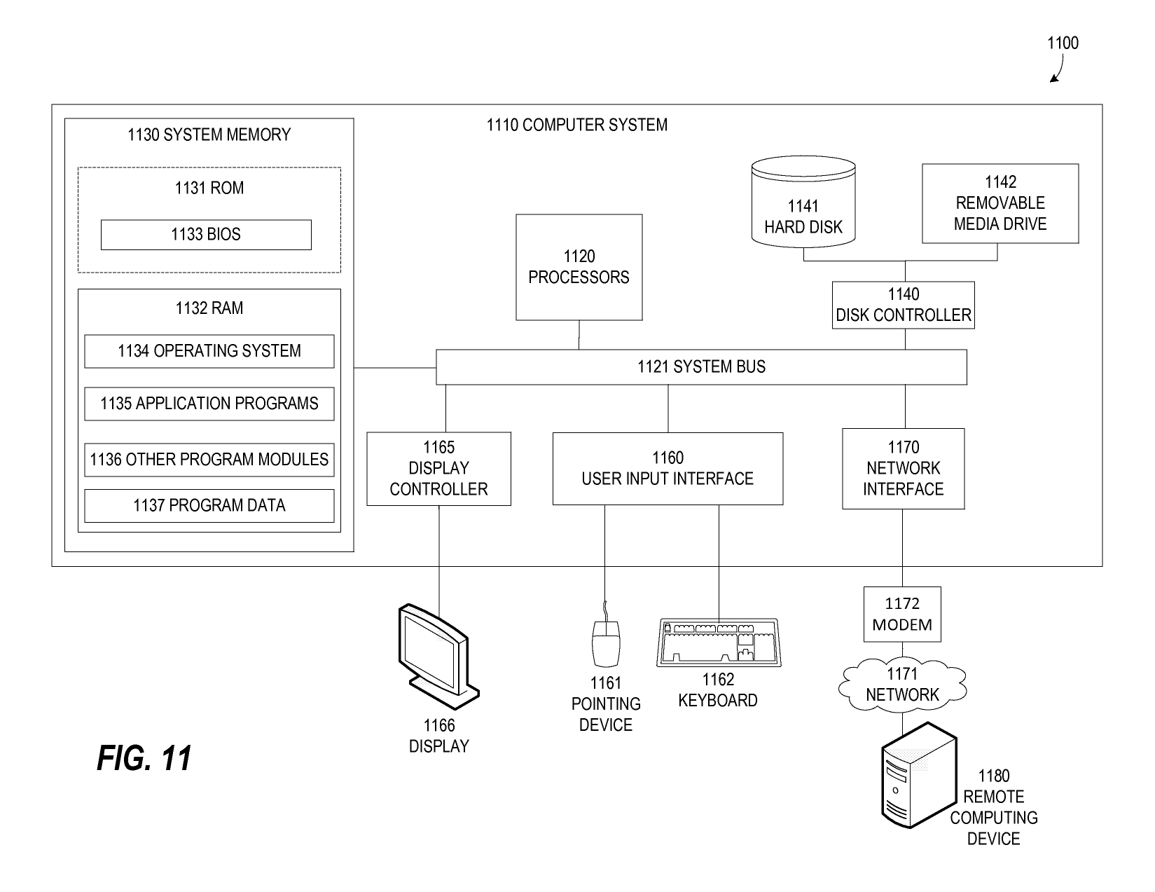

FIG. 11 illustrates an example of a computing environment 1100 within which embodiments of the invention may be implemented. Computing environment 1100 may include computer system 1110, which is one example of a computing system upon which embodiments of the invention may be implemented. As shown in FIG. 11, the computer system 1110 may include a communication mechanism such as a bus 1121 or other communication mechanism for communicating information within the computer system 1110. The system 1110 further includes one or more processors 1120 coupled with the bus 1121 for processing the information. The processors 1120 may include one or more CPUs, GPUs, or any other processor known in the art.

The computer system 1110 also includes a system memory 1130 coupled to the bus 1121 for storing information and instructions to be executed by processors 1120. The system memory 1130 may include computer readable storage media in the form of volatile and/or nonvolatile memory, such as read only memory (ROM) 1131 and/or random access memory (RAM) 1132. The system memory RAM 1132 may include other dynamic storage device(s) (e.g., dynamic RAM, static RAM, and synchronous DRAM). The system memory ROM 1131 may include other static storage device(s) (e.g., programmable ROM, erasable PROM, and electrically erasable PROM). In addition, the system memory 1130 may be used for storing temporary variables or other intermediate information during the execution of instructions by the processors 1120. A basic input/output system 1133 (BIOS) containing the basic routines that help to transfer information between elements within computer system 1110, such as during start-up, may be stored in ROM 1131. RAM 1132 may contain data and/or program modules that are immediately accessible to and/or presently being operated on by the processors 1120. System memory 1130 may additionally include, for example, operating system 1134, application programs 1135, other program modules 1136 and program data 1137.

The computer system 1110 also includes a disk controller 1140 coupled to the bus 1121 to control one or more storage devices for storing information and instructions, such as a magnetic hard disk 1141 and a removable media drive 1142 (e.g., floppy disk drive, compact disc drive, tape drive, and/or solid state drive). The storage devices may be added to the computer system 1110 using an appropriate device interface (e.g., a small computer system interface (SCSI), integrated device electronics (IDE), Universal Serial Bus (USB), or FireWire).

The computer system 1110 may also include a display controller 1165 coupled to the bus 1121 to control a display or monitor 1166, such as a cathode ray tube (CRT) or liquid crystal display (LCD), for displaying information to a computer user. The computer system includes an input interface 1160 and one or more input devices, such as a keyboard 1162 and a pointing device 1161, for interacting with a computer user and providing information to the processor 1120. The pointing device 1161, for example, may be a mouse, a trackball, or a pointing stick for communicating direction information and command selections to the processor 1120 and for controlling cursor movement on the display 1166. The display 1166 may provide a touch screen interface which allows input to supplement or replace the communication of direction information and command selections by the pointing device 1161.

The computer system 1110 may perform a portion or all of the processing steps of embodiments of the invention in response to the processors 1120 executing one or more sequences of one or more instructions contained in a memory, such as the system memory 1130. Such instructions may be read into the system memory 1130 from another computer readable medium, such as a hard disk 1141 or a removable media drive 1142. The hard disk 1141 may contain one or more datastores and data files used by embodiments of the present invention. Datastore contents and data files may be encrypted to improve security. The processors 1120 may also be employed in a multi-processing arrangement to execute the one or more sequences of instructions contained in system memory 1130. In alternative embodiments, hard-wired circuitry may be used in place of or in combination with software instructions. Thus, embodiments are not limited to any specific combination of hardware circuitry and software.

As stated above, the computer system 1110 may include at least one computer readable medium or memory for holding instructions programmed according to embodiments of the invention and for containing data structures, tables, records, or other data described herein. The term “computer readable medium” as used herein refers to any non-transitory, tangible medium that participates in providing instructions to the processor 1120 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 1141 or removable media drive 1142. Non-limiting examples of volatile media include dynamic memory, such as system memory 1130. Non-limiting examples of transmission media include coaxial cables, copper wire, and fiber optics, including the wires that make up the bus 1121. Transmission media may also take the form of acoustic or light waves, such as those generated during radio wave and infrared data communications.

The computing environment 1100 may further include the computer system 1110 operating in a networked environment using logical connections to one or more remote computers, such as remote computer 1180. Remote computer 1180 may be a personal computer (laptop or desktop), a mobile device, a server, a router, a network PC, a peer device or other common network node, and typically includes many or all of the elements described above relative to computer 1110. When used in a networking environment, computer 1110 may include modem 1172 for establishing communications over a network 1171, such as the Internet. Modem 1172 may be connected to system bus 1121 via user network interface 1170, or via another appropriate mechanism.

Network 1171 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 1110 and other computers (e.g., remote computing system 1180). The network 1171 may be wired, wireless or a combination thereof. Wired connections may be implemented using Ethernet, Universal Serial Bus (USB), RJ-11 or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 1171.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A graphical user interface (GUI), as used herein, comprises one or more display images, generated by a display processor and enabling user interaction with a processor or other device and associated data acquisition and processing functions.

The GUI also includes an executable procedure or executable application. The executable procedure or executable application conditions the display processor to generate signals representing the GUI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the GUI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

The system and processes of the figures presented herein are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. Further, the processes and applications may, in alternative embodiments, be located on one or more (e.g., distributed) processing devices on a network linking the units of FIG. 11. Any of the functions and steps provided in the Figures may be implemented in hardware, software or a combination of both. No claim element herein is to be construed under the provisions of 35 U.S.C. 112, sixth paragraph, unless the element is expressly recited using the phrase “means for.”

The embodiments of the present disclosure may be implemented with any combination of hardware and software. In addition, the embodiments of the present disclosure may be included in an article of manufacture (e.g., one or more computer program products) having, for example, computer-readable, non-transitory media. The media has embodied therein, for instance, computer readable program code for providing and facilitating the mechanisms of the embodiments of the present disclosure. The article of manufacture can be included as part of a computer system or sold separately.

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

1. A steerable body insertion device comprising:
a bendable non-piezoelectric element configured to move within patient anatomy, the non-piezoelectric element extending an element length between a proximal end and a distal end and having an element center axis extending along the element length when the non-piezoelectric element is in a non-bent state; and
a first piezoelectric strand coupled to a surface of the non-piezoelectric element and extending a first strand length, the first piezoelectric strand having a first strand center axis extending substantially parallel to the element center axis along the first stand length;
wherein when a first voltage is applied to the first piezoelectric strand, the first piezoelectric strand is configured to contract and cause the non-piezoelectric element to bend away from the element center axis.

2. The steerable body insertion device according to claim 1, wherein the bendable non-piezoelectric element is a pull wire and the first piezoelectric strand is coupled to a pull wire surface at the distal end.

3. The steerable body insertion device according to claim 1, wherein the bendable non-piezoelectric element is a catheter and the first piezoelectric strand is coupled to a catheter surface.

4. The steerable body insertion device according to claim 1, further comprising:
a second piezoelectric strand coupled to the non-piezoelectric element, opposing the first piezoelectric strand, having a second strand length, and having a second strand center axis extending along the second stand length and substantially parallel to the element center axis, and
wherein,
when a first voltage is applied to the first piezoelectric strand, the first piezoelectric strand is configured to contract and cause the non-piezoelectric element to bend away from the element center axis in a first direction toward the first piezoelectric strand relative to the element axis, and
when a second voltage is applied to the second piezoelectric strand, the second piezoelectric strand is configured to contract and cause the non-piezoelectric element to bend away from the element center axis in a second direction toward the second piezoelectric strand relative to the element center axis, the second direction being opposite the first direction.

5. The steerable body insertion device according to claim 1, further comprising:
a second piezoelectric strand coupled to the surface of the bendable non-piezoelectric element and spaced from the first piezoelectric strand, the second piezoelectric strand extending a second strand length and having a second strand center axis extending substantially parallel to the element center axis along the second stand length,
wherein when a second voltage is applied to the second piezoelectric strand simultaneously with the first voltage applied to the first piezoelectric strand, the first piezoelectric strand and the second piezoelectric strand are each configured to contract and cause the bendable non-piezoelectric element to bend away from the element center axis in a third direction, the third direction having directional components in the first direction and the second direction.

6. The steerable body insertion device according to claim 1, further comprising:
a second piezoelectric strand coupled to the surface of the bendable non-piezoelectric element and spaced from the first piezoelectric strand, the second piezoelectric strand extending a second strand length and having a second strand center axis extending substantially parallel to the element center axis along the second stand length; and
a third piezoelectric strand coupled to the surface of the bendable non-piezoelectric element and spaced from the first and second piezoelectric strands, the third piezoelectric strand extending a third strand length and having a third strand center axis extending substantially parallel to the element center axis along the third stand length,
wherein when a second voltage is applied to the second piezoelectric strand, the second piezoelectric strand is configured to contract and cause the bendable non-piezoelectric element to bend away from the element center axis in a second direction, the second direction being different than the first direction, and
wherein when a third voltage is applied to the third piezoelectric strand, the third piezoelectric strand is configured to contract and cause the bendable non-piezoelectric element to bend away from the element center axis in a third direction, the third direction being different than the first direction and the second direction.

7. The steerable body insertion device according to claim 1, wherein the amount that the bendable non-piezoelectric element bends away from the element center axis is proportional to the magnitude of the first voltage applied to the first piezoelectric strand.

8. A steerable body insertion device comprising:
a bendable non-piezoelectric element configured to move within patient anatomy, the bendable non-piezoelectric element extending an element length between a proximal end and a distal end, the bendable non-piezoelectric element having an element center axis extending the element length when the non-piezoelectric element is in a non-bent element state; and
one or more piezoelectric strands embedded within the non-piezoelectric element, the one or more piezoelectric strands extending a strand length between corresponding strand ends and having a corresponding strand center axis extending substantially parallel to the element center axis along the strand length when the corresponding piezoelectric strand is in a non-bent strand state,
wherein when a voltage is applied to the one or more piezoelectric strands, the one or more piezoelectric strands are configured to contract and cause the non-piezoelectric element to bend away from the element center axis in a direction toward the one or more piezoelectric strands relative to the element center axis.

9. The steerable body insertion device according to claim 8, further comprising a plurality of piezoelectric strands each having a corresponding strand center axis that is spaced equidistant from the element center axis, and
when the voltage is applied to the one or more piezoelectric strands, the one or more piezoelectric strands are configured to contract and cause the non-piezoelectric element to bend away from the element center axis in a direction toward the one or more piezoelectric strands relative to the element center axis.

10. The steerable body insertion device according to claim 9, further comprising an inner lumen portion extending along the element length, wherein
the non-piezoelectric element comprises an outer portion at least partially housing the inner lumen portion,
each of the plurality of piezoelectric strands of the combined piezoelectric strand set are spaced from each other and disposed within the outer portion, and
when the voltage is applied to the one or more piezoelectric strands, the one or more piezoelectric strands are configured to contract and cause the outer portion of the non-piezoelectric element to bend away from the element center axis in a direction toward the one or more piezoelectric strands relative to the element center axis.

11. The steerable body insertion device according to claim 10, wherein the plurality of piezoelectric strands comprise a first piezoelectric strand, a second piezoelectric strand and a third piezoelectric strand each having a corresponding strand center axis spaced equidistant from each other.

12. The steerable body insertion device according to claim 8, wherein the bendable non-piezoelectric element is a portion of a sheath.

13. The steerable body insertion device according to claim 8, wherein the bendable non-piezoelectric element is a portion of a catheter.

14. The steerable body insertion device according to claim 8, further comprising a first piezoelectric strand and a second piezoelectric strand, and
when a first voltage is applied to the first piezoelectric strand simultaneously with a second voltage applied to the second piezoelectric strand, the first piezoelectric strand and the second piezoelectric strand are each configured to contract and cause the bendable non-piezoelectric element to bend away from the element center axis in a direction toward the one or more piezoelectric strands relative to the element axis, the direction having directional components in a first direction toward the first piezoelectric strand relative to the element axis and a second direction toward the second piezoelectric strand relative to the element center axis.

15. The steerable body insertion device according to claim 8, wherein the amount that the bendable non-piezoelectric element bends away from the element center axis is based on the magnitude of the voltage applied to the one or more piezoelectric strands.

16. The steerable body insertion device according to claim 8, further comprising multiple sets embedded and substantially centered within the non-piezoelectric element extending along the element length.

17. The steerable body insertion device according to claim 8, wherein the one or more piezoelectric strands comprises a plurality of electrodes to separate the one or more piezoelectric strands into a plurality of sub-piezoelectric strands between the electrodes, each of the one or more sub-piezoelectric strands configured to contract and cause the non-piezoelectric element to bend away from the element center axis in a direction toward the one or more sub-piezoelectric strands relative to the element center axis when the voltage is applied to a corresponding sub-piezoelectric strand.

18. The steerable body insertion device according to claim 8, wherein a first strand length of a first piezoelectric strand is different than a second strand length of a second piezoelectric strand.

19. A system for controlling a steerable body insertion device, the system comprising:
a steerable body insertion device comprising:
a bendable non-piezoelectric element configured to move within patient anatomy, the bendable non-piezoelectric element extending an element length between a proximal end and a distal end, the bendable non-piezoelectric element having an element center axis extending the element length when bendable the non-piezoelectric element is in a non-bent element state; and
a plurality of piezoelectric strands coupled to the non-piezoelectric element, each of the plurality of piezoelectric strands extending a strand length between corresponding strand ends and having a corresponding strand center axis extending substantially parallel to the element center axis along the strand length when the corresponding piezoelectric strand is in a non-bent strand state, each of the plurality of piezoelectric strands configured to contract when receiving a voltage and cause the non-piezoelectric element to bend away from the element center axis in a direction toward one or more of the plurality of piezoelectric strands relative to the element center axis;
a voltage applicator configured to apply the voltage to the one or more piezoelectric strands; and
a controller configured to control the bend of the bendable non-piezoelectric element by causing the voltage applicator to apply the voltage to the one or more piezoelectric strands.

20. The system according to claim 19, wherein the controller is further configured to control an amount of the bend of the non-piezoelectric element by controlling the voltage applicator to apply a voltage magnitude to the one or more piezoelectric strands.

21. The system according to claim 19, wherein the controller is further configured to control the direction of the bend of the non-piezoelectric element by controlling the voltage applicator to apply a voltage to the one or more piezoelectric strands.

22. The system according to claim 19, wherein
the voltage applicator is further configured to apply a first voltage to a first piezoelectric strand and simultaneously apply a second voltage to a second piezoelectric strand;
the controller is further configured to cause the non-piezoelectric element to bend in a direction having directional components in a first direction toward the first piezoelectric strand relative to the element axis and a second direction toward the second piezoelectric strand relative to the element center axis.

23. The system according to claim 22, wherein the controller is further configured to cause the magnitude of the second voltage applied to the second piezoelectric strand to be different than the first voltage applied to the first piezoelectric strand.

24. A piezoelectric strand set for use with a non-piezoelectric element configured to move within patient anatomy, the piezoelectric strand combination comprising:
a plurality of piezoelectric strands extending a corresponding strand length between corresponding strand ends and having a corresponding strand center axis extending along the corresponding strand length when the plurality of piezoelectric strands piezoelectric strands are in a non-bent state and each of the plurality of piezoelectric strands strand having opposing electrical contacts electrically connected at opposite ends of each strand configured to receive an applied voltage,
wherein each of the plurality of piezoelectric strands are configured to be embedded into the non-piezoelectric element, and
when the voltage is applied to one or more of the plurality of piezoelectric strands, the one or more piezoelectric strands are configured to contract and cause the non-piezoelectric element to bend away from a center axis of the non-piezoelectric element in a direction toward the one or more piezoelectric strands relative to the center axis of the non-piezoelectric element.
